(19) United States
(12) Patent Application Publication
(10) Pub. No.: US 2003/0079240 A1
(43) Pub. Date: Apr. 24, 2003

(54) Chimeric embryos and animals containing human cells

(76) Inventor: Stuart A. Newman, Pleasantville, NY (US)

Correspondence Address: COLLIER SHANNON SCOTT, PLLC, 3050 K STREET, NW, SUITE 400, WASHINGTON, DC, 20007, US

(21) Appl. No.: 10/308,135

(22) Filed: Dec. 3, 2002

Related U.S. Application Data
(62) Division of application No. 08/993,564, filed on Dec. 18, 1997.

(51) Int. Cl. A01K 67/00

(52) U.S. Cl. 800/8

(57) ABSTRACT

A mammalian embryo developed from a mixture of embryo cells, embryo cells and embryonic stem cells, or embryonic stem cells exclusively, in which at least one of the cells is derived from a human embryo, a human embryonic stem cell line, or any other type of human cell, and any cell line, developed embryo, or animal derived from such an embryo.

### FIELD OF THE INVENTION

This invention relates to chimeric embryos and animals. More specifically, the invention relates to chimeric embryos and chimeric animals created from human embryos or embryonic stem (ES) cells and embryos or ES cells from one or more non-human animals, which have been aggregated under conditions in which a viable embryo forms. Chimeric embryos are embryos derived from cells whose origin is in two different species, or in two different strains or genotypes of a single species. Chimeric animals are any animals resulting from the development of chimeric embryos. The availability of chimeric embryos and animals containing human cells will facilitate the study of human cellular development, improve our ability to assay and develop new therapeutics, and advance the study of organ and tissue transplantation, as well as enable other advances in the science.

### BACKGROUND OF THE INVENTION

Based on our current state of knowledge, chimeric embryos are generated by using any of three currently available technologies. Although these technologies represent the current state of the art, the present invention anticipates that additional technologies, or modifications to current technologies, may be developed to create chimeric embryos. The first technology aggregates cells derived from the early embryos of two or more different animal species or strains. These cells, will, under favorable circumstances, remain attached to one another and cooperate in the formation of a more developed embryo, then a juvenile, and ultimately an adult organism exhibiting features in common with, and different from, individuals of the species or strains from which the embryo cells originated. Fehilly et al. (1984) Nature, 307, 634-636 and Meinecke-Tillmann and Meinecke (1984) Nature, 307 637-638, reported the experimental formation of sheep-goat chimeric embryos, and birth of chimeric “geeps” after transplantation to sheep or goat mothers.

The Fehilly, et al. study described three individual methods to create chimeric embryos which further define, and are a part of, the first technology described above. The three methods were employed in an attempt to create the sheep-goat chimeras. In the first method, single blastomeres from four-cell goat embryos were combined with single blastomeres from four-cell sheep embryos, or with single blastomeres from eight-cell sheep embryos in evacuated zonae pellucida as described in S. M. Willadsen (1979) Nature, 277 298-300.

In the second method of the first technology, interspecific embryos were produced by either surrounding an eight-cell goat embryo from which the zona pellucida had been removed with the dissociated blastomeres of three eight-cell sheep embryos or by surrounding a similarly denuded eight-cell sheep embryo with the separated blastomeres of three eight-cell goat embryos. The embryos resulting from the first two methods were embedded in agar and cultured in ligated sheep oviducts for four or five days, depending upon the particular experiment. Those embryos which developed into normally organized chimeric blastocysts, were then transferred to recipient ewes or recipient goats.

In the third method of the first technology, the inner cell mass and polar trophectoderm from day eight goat blastocysts were inserted into day eight sheep blastocysts, and vice-versa, by the technique described by R. L. Gardner (1968) Nature, 220, 596-597. The blastocysts were then transferred to sheep or goat recipients.

These experiments demonstrated that sheep and goat blastomeres can form chimeric blastocysts and that such inter-species blastocysts are viable and may give rise to animals which are sheep-goat chimeras. The experiments also demonstrate that a goat fetus can develop to term in a sheep, and a sheep fetus can develop to term in a goat.

Meinecke-Tillmann and Meinecke used the first technology to create interspecific sheep-goat chimeric embryos. The embryos were created by combining one sheep four-cell blastomere with two goat eight-cell blastomeres, or by combining two sheep eight-cell blastomeres with two goat eight-cell blastomeres, in a common pig zona pellucida. Micromanipulation of the blastomeres was performed as described by S. Meinecke-Tillmann and B. Meinecke (1983) Zentbl. VetMed., 30, 146-153. The embryos which continued to develop were transferred to the oviducts of an intermediate recipient and embryos which reached the blastocyst stage were then transferred to the uterine horns of the final sheep recipients.

These studies determined that chimeric animals could be brought to term in different species when there is a protective mechanism which prevents recognition of the foreign fetus by the mother of the other species. This protection may result from the formation of a protective population of cells, i.e., the chorion epithelium. The chorion epithelium is developed from the trophoblast of the chimeric blastocyst, and is derived from the embryonic component of the chimeric blastocyst of the same species as the mother. For example, if the chorion epithelium was derived from the trophoblast which developed from the sheep blastomere, the sheep trophoblast components were able to protect the goat embryonic cells from the sheep maternal immune rejection. Meinecke-Tillmann and Meinecke suggest that the ability to create chimeric embryos, and bring them to term as chimeric animals in interspecific hosts, could be useful for rescuing endangered species.

The second technique for generating chimeric embryos is through the use of embryonic stem (ES) cells. ES cells are undifferentiated, immortal cells. They are derived from the inner cell mass (ICM) of preimplantation mammalian embryos, by culturing these embryo cells under defined conditions. These cells are totipotent, i.e., capable of differentiating into derivatives of all three of the basic embryonic germ layers, from which all cell types ultimately develop. Martin (1981) Proc. Nat. Acad. Sci. USA 78, 7634-7638, described the establishment of an ES cell line from the mouse embryo. It was not until recently, however, that Thomson et. al. (1995) Proc. Nat. Acad. Sci. USA 92, 7844-7848, described the isolation of an ES cell line from the embryo of the rhesus monkey. ES cells have the ability to remain undifferentiated and proliferate indefinitely in vitro while maintaining the potential to differentiate into derivatives of all three embryonic germ layers. Thomson suggests that the use of human ES cells would offer “exciting new possibilities for transplantation medicine.” (Thomson et. al. (1995) Proc. Nat. Acad. Sci. USA 92, at 7848.) When combined with normal preimplantation embryos of the same or different strain from which they were derived, ES cells participate in normal development, potentially contributing cells to the tissues of the resulting animal (Bradley et. al. (1984) Nature, 309, 255-256). Because ES cells can be propagated in culture, and experimentally manipulated, their use in chimeric embryos affords the possibility of introducing “transgenes”. Transgenes are genes originating from other species or individuals. These manipulations may serve to reduce immunogenicity or to give the cells additional functionalities to combat specific diseases. ES cells are now widely used for the introduction of specific, targeted mutations and other genetic alterations, as has been shown in the mouse germ line by Rossant, et al. (1993) Philos. Trans. R. Soc. London B, 339, 207-215 and Koller, et al. (1992) Annu. Rev. Immunol., 10 705-730.

The third technique for generating chimeric embryos is through the use of “early passage” ES cells, i.e. cells that have been permitted only a few divisions in culture after the ES cell line is established, or certain ES cell subclones that retain totipotency at later passages. These cells are aggregated with defective embryos genetically incapable of advancing beyond the early stages of development, but which provide components that mediate implantation of the chimeric cell aggregates. Using this technique, Nagy et al. (1993) Proc. Nat. Acad. Sci. 90, 8424-8428, generated viable, normal, fertile mice, which were completely ES-cell derived. The technique is based on the aggregation of ES cells with developmentally compromised tetraploid embryos. In such chimeras the tetraploid is selected against and ES cells differentiate normally, to form viable embryos. ES cells have proved to be an efficient vehicle for transmitting genetic manipulations into the germ line after either injection into blastocysts or aggregation with diploid eight-cell stage embryos in the mouse. Nagy, et al. suggest that this technique may help to advance the technology of genetic manipulation of the mammalian genome. Chimeric embryos would result from using this procedure with a mixture of ES cells derived from different strains or species. The use of this technology, as an object of the present invention, will permit the further study of embryonic development disorders by creating embryos with interspecific ES cells.

Chimeric embryos consisting exclusively of ES cells may also incorporate transgenes, which are introduced into the ES cells prior to aggregation. Transgenes may be introduced using standard methods well-known in the art. Hermiston et al. (1993) Proc. Nat. Acad. Sci. 90, 8866-8870, for example, produced chimeric-transgenic mice using mouse embryo and ES cells. The model was developed to examine the effects of wild-type or mutant proteins on cell fate determination, and the effect of these proteins on the proliferation and differentiation programs of cells. In these experiments, ES cells were transfected with recombinant DNAs of known function, and the stably transfected ES cells are subsequently introduced into host mouse blastocysts to create chimeric-transgenic mice. Adult mice are formed which possess specific cells which are derived from both the host and the transgenic-chimeric cells.

In this way one animal may be used to study the effect of both cell types, normal and transgenic. Interspecific chimeric embryos, as an object of the present invention, can be used to study the effect of the introduction of a larger number of foreign genes and gene products on the development and function of predetermined populations of cells. In addition, the interspecific chimeric embryos may be used in the study of the effects of large numbers of genes or gene products on the biological properties of specific cell populations. The use of such a technology in the present invention would permit the development of human/non-human mammalian models, where chimeras contain a number of human genes in a specific cell type. These models may be used for the study of normal cellular development, or the development of certain disease states.

Once chimeric embryos are produced they can be propagated for varying periods of time in culture, where they may undergo a series of developmental steps. For methods of culturing mammalian embryos, see Paria et al. (1992) Proc. Nat Acad. Sci. USA 89 10051-1055; Burdsal et al. (1993) Development 118, 829-844; and Rasmussen et al. (1994) Teratology 49, 20-28. For some uses, the embryos can be brought to term, forming the chimeric animals of the invention.

In the case of mammalian chimeric embryos, progression through later stages, and completion of development to term, requires implantation and placentation, and therefore introduction into the uterus of a hormonally prepared “pseudopregnant” female. The foster mother can be of the same species as the embryo or one of its cellular components, but need not be. Summers et al. (1987) J. Reprod. Fertil. 80, 13-20, performed successful trans-species embryo transfers, introducing preimplantation zebra embryos into the uteri of domestic horses, where they underwent further development and gave rise to normal zebra foals. The authors of this study note that chimeric embryos, like their non-chimeric counterparts, can be cryopreserved for later use.

It will be apparent to those skilled in the art that various modifications and variations can be made in the manner in which the chimeric embryo is created and propagated, without departing from the scope or spirit of the present invention. For example, substantial research is currently in progress toward the development of artificial uteri for various species, both mammalian and non-mammalian. In addition, various in vitro techniques have been and continue to be investigated that could potentially sustain life in vitro and allow for the propagation and further development of embryonic cells. By means of further example, alternatives to the three methods of preparing the embryo discussed above may also be developed. Thus, it is intended that the present invention cover all of the modifications and variations of the invention, provided they come within the scope of the appended claims and their equivalents.

One use of chimeric embryos containing human cells would be in the scientific field of developmental biology, where it is of interest to determine the factors which regulate the differentiation of human embryonic cells, and their assembly into tissues and organs. Social and legal constraints prevent the observation of, and experimentation on, human embryos at advanced stages of development. In contrast, chimeric embryos containing human cells can be followed in culture, or harvested from the uteri of non-human foster mothers, and subjected to histological and experimental analysis. The human cell contribution to various cells and organs can be determined using species-specific antibody labeling techniques, or using the technique described by Hermiston et al. in their study of intestinal cell development in mouse/mouse chimeras. The human cells can be transfected with a “reporter gene” by which their fate can be followed during subsequent development.

Chimeric embryos containing human cells will be useful in the fields of teratology and toxicology. For example, Uno et al. (1990) Brian Res. Dev. Brian Res. 53, 157-167, found that the drug dexamethasone, administered to pregnant rhesus monkeys, caused brain damage to the developing fetuses, as evidenced by degeneration of specific subpopulations of neurons. The study was designed and conducted to mimic human clinical trials. Up until the present invention, such trials could not be performed on tissues containing a human cellular component. Using fetal brains containing human cells (which could be identified by specific antibody labeling), the present inventor believes that it could be determined whether developing human neurons were correspondingly susceptible to dexamethasone or other drugs.

One of the objects of the present invention is to create model systems to permit the clinical testing, or simple assays, of compounds on human cells, without risk to human subjects. It is well known in the art that many drugs that are highly efficacious in animal models, may have little or no effect in humans. Unfortunately, some of these compounds may actually have deleterious effects when administered to humans. It is an important object of the invention to create model systems which will not place humans at risk during clinical testing of these compounds.

Many drugs available today, and many compounds in drug development, suffer from the “risk versus benefit” dilemma. The compound has proven efficacy, but also possesses unavoidable, potentially serious, side effects. It is important to determine at which dose the risk that the compound poses is greater than the benefit it provides. Many of the “risk versus benefit” drugs have relied on inadequate animal models to determine the “risk” factor of the equation. The present invention allows a more accurate determination of the compound's effect on human cells and/or human tissue without the need to expose human subjects to trials with new and unknown compounds. An additional advantage of the present invention is the ability to more accurately determine the proper dose rate in human cells and/or tissues of the high risk compounds.

Chimeric embryos and animals containing human cells would be useful in the scientific field of physiology, in which response of tissues to various stimuli and treatments is studied. For example, McAuliffe and Robbins (1991) Pediatr. Res. 29, 580-585, studied the effect of pressure overload in the fetal sheep heart on the production of a protein involved in cardiac hypertrophy. McAuliffe and Robbins concede that the change in the pattern of expression of certain proteins of the myocardium varies from animal to animal. Little or no correlation can be drawn from the results of many studies of heart muscle between man and currently available animal models. Studies similar to those conducted by McAuliffe and Robbins on fetal hearts containing human cells (an object of the present invention) could determine patterns of human cardiac protein expression in response to stress. The development of chimeras containing human heart muscle are model systems for the assay of known beneficial and/or unknown compounds for their effect on the treatment of heart muscle disorders.

In another study, Knowlton et al. (1991) J. Clin. Invest. 88 2018-2025, used balloon catheterization of the isolated adult rabbit heart to determine the role of ventricular stretch on the production of a protein associated with tissue stress. Hearts containing human cells, would provide as yet unobtainable information on the ability of these cells to produce stress proteins in response to similar conditions. Protein production in heart tissue has been shown to be highly variable among species. The present invention is an invaluable model in the study the effects of various stimuli on human heart tissue.

Chimeric organisms would also find use in the field of transplantation medicine, which suffers from a shortage of donor organs. Although attempts have been made to transplant baboon hearts and bone marrow into humans, and pig hearts into primates, rejection is usually swift unless complex drug treatment protocols accompany the transplantation, or genetically modified organs are used. Even these procedures can only guarantee limited success at the present time (Bach et al. (1995) Nature Medicine 1, 869-873).

The present invention enables one to genetically engineer donor animals. In recent studies involving pig donor organs into primates, the xenografts have undergone dramatic hyperacute rejection within minutes to one to two hours upon transplantation into untreated recipients. (Bach, F. H. et al. (1991), Transplantatn. Proc. 23, 205-208 and Bach, F. H. et al. (1994), Immunol. Rev., 141, 5-30.) These studies suggest that endothelial cell (EC) actuation underlies the vascular aspects of xenograft rejection. Use of host endothelial progenitor cells in the chimeric embryo of the present invention will be used to target this actuation process and negate its consequences which may contribute to xenograft rejection. It has been shown in vitro that supplying a particular human protein to pig endothelial cells blocked human complement, and in turn blocked human serum from destroying the transplanted pig cells. (Bach, F. H. et al. (1994), Immunol. Rev., 141, 5-30 and Dalmasso, A. P. et al. (1991), Transplantation, 52, 530-533.) Current genetic therapies for xenotransplantation include the production of transgenic pigs which express human complement inhibitors. (Cozzi, E. and White, D. J. G., (1995) Nature Med. 1, 964-966.) The present invention will allow the creation of tissues which could be transplanted across species lines without the need to block or otherwise inactivate host pathways. Chimeric tissues overcome major immunological barriers during their development (Fehilly et al., 1984; Meinecke-Tillmann and Meinecke, 1984). They are often tolerated as grafts by non-chimeric members of the founder species (Gustafson et al., 1993). Tissues created by the present invention represent promising sources of tissues and organs for xenotransplantation (transplantation across species lines).

Production of chimeric animals proved capable of reducing immunological incompatibilities between the tissues of different species. Gustafson et al. (1993) J. Reprod. Immunol. 23, 155-168, found that some normal sheep and goat siblings of sheep-goat chimeras were able to tolerate skin grafts from their chimeric siblings and exhibited immune tolerance to their chimeric siblings as measured by the mixed lymphocyte response (MLR). The results of this and related studies demonstrate the potential for the development of chimeric animals to be used as a source of tissue for skin grafts.

The importance of creating an animal model system where multiple proteins are derived from one of the chimeric species is illustrated in the study by Bach, F. H. et al., (1995), Nature Medicine 1, 869-873. It is well known in the art that an expected effect may not be realized if only a single transgene is expressed in the chimeric individual. Many biological processes are dependent upon the interaction of multiple proteins. If the protein encoded by the transgene does not effectively interact with the host's complementary protein or proteins, the desired biological effect will not occur. Bach, et al. refer to this phenomenon as “molecular incompatibility”. Bach also suggests that the effect may not only be negative in function, but the interaction between related proteins of different species may initiate reactions that might not ordinarily take place in a non-chimeric organism.

For these reasons it is important to supply as many genes as possible from the animal under investigation into the host to form the chimera. This effect cannot be accomplished by specifically engineering one gene at a time. As such, it is important to ensure that multiple proteins in a related pathway are expressed from the same species. An important object of the present invention may allow the development of chimeric animals in which multiple foreign genes are expressed in the chimera, not only one or two specifically engineered transgenes.

For example, there are multiple cytokines that will not function across species lines and it may be critical that the entire family of cytokines be synthesized by cells originating from the same species. The ability to express a family of genes which control immunorejection from one cell type in the chimera may advance technology and facilitate the ability to donate organs across species. The acceptance or rejection of donor organs is known to be based upon the inter-relationship of multiple factors. It is critical that systems be devised to ensure the expression of entire families of inter-related genes. The present invention provides a model for developing such systems.

Current studies have described the creation of chimeric embryos using non-human animals as donor and recipient cells. These chimeras are useful for limited studies on the development of animal tissues and organs, but do not address the complex processes associated with the development of human cells. The use of animal cells allows only generalizations about the specific developmental potential of human cells. The present invention solves these problems by specifically incorporating human cells into the developing chimera in order to study human cellular development. Only by studying the actual development of human tissues and organs can we understand the disorders that affect human development and use this knowledge to discover methods to repair and/or cure human developmental disorders.

In addition, the development of chimeras containing human cells permits more reliable, extensive, and conclusive clinical trials than available using solely animal models in the development of new therapeutic agents. Trials using human/non-human chimeras would not expose humans to risk, and would also decrease the number of experimental animals needed to conduct these trials. The ability to use chimeras to more thoroughly and safely assess the risk and benefits of new compounds would ultimately decrease the time it takes to get new therapeutics to market, and will also decrease the cost of developing these new drugs.

The ability to develop the technology of the present invention is crucial for the continued discovery and development of experimental new drugs and therapies to treat existing and as yet unknown diseases. The ability to provide research models which are safe, reliable, and decrease the need for risky human and animal trials is an important object of the present invention.

Current studies have attempted to create organs and tissues for transplantation into humans. The creation of non-human chimeras for this purpose has met with limited success. The ability to create human chimeras as a source for human tissue and ultimately human organs would overcome the current difficulties with immunorejection of foreign tissue. The ability to have an unlimited supply of tissue for skin grafts and organs for transplant would save many lives and greatly increase the quality of life of many.

### OBJECTS OF THE INVENTION

It is therefore an object of the present invention to create chimeric mouse/human embryos for studies in developmental biology.

It is another object of the present invention to create chimeric baboon/human or chimpanzee/human embryos for developmental toxicology assays.

It is a further object of the present invention to create chimpanzee/human chimeras for studies in cardiovascular physiology.

It is still another object of the present invention to create baboon/human chimeras as a source of bone marrow for transplantation.

It is yet another object of the present invention to create chimpanzee/human and pig/human chimeras as a source of hearts for transplantation to cardiac patients.

It is another object of the present invention to create human/non-human chimeras for the study of embryonic development disorders.

It is a further object of the present invention to create human/non-human chimeric embryos which can be cryopreserved for later use.

It is still another object of the present invention to create human/non-human chimeras to be used as a source of tissue for skin grafts.

It is yet another object of the present invention to create human/non-human chimeras to be used as a source of organs for transplants.

It is another object of the present invention to create human/non-human chimeras for use as model systems for use in research and in clinical trials.

It is a further object of the present invention to create human/non-human chimeras for use in clinical trials which will decrease the number of human and animal subjects required for such trials.

It is still another object of the present invention to create human/non-human chimeras which would streamline the drug research and development process.

It is yet another object of the present invention to create human/non-human chimeras which would make the drug development process less costly.

Additional objects and advantages of the invention are set forth, in part, in the description which follows and, in part, will be apparent to one of ordinary skill in the art from the description and/or from the practice of the invention.

### SUMMARY OF THE INVENTION

In response to the foregoing challenge, Applicants have developed a strategy to create innovative chimeric embryos, cell lines, and animals for use in research, medicine, drug development, and disease prevention. Applicants have also developed a strategy to create chimeric embryos, cell lines, and animals which can be used as organ and/or tissue donors for other animals to include humans.

The invention comprises, in part, human embryos, human embryonic cells, and/or human embryonic stem cells and embryos, embryonic cells, and/or embryonic stem cells from one or more non-human animal species, which have been aggregated under conditions in which a viable embryo forms. The human embryo cells may be obtained from any number of sources, to include, but not limited to, superfluous newly-generated embryos at in vitro fertilization clinics, or from cryopreserved embryos for which the decision has been made to not bring them to term. Human embryonic stem cells may be generated in any number of ways, to include, but not limited to, the methods used by Thomson et al. to produce embryonic stem cells from another primate species. Embryos of mice, rats, rabbits, pigs, rhesus monkeys, macaques, chimpanzees, and other mammalian species may be obtained from any number of sources, including, but not limited to, breeding populations resulting from standard laboratory and animal husbandry methods. In addition, embryonic stem cell lines are available for some of these species.

More specifically, the present invention is a chimeric embryo comprising cells from two or more different animal species, wherein one or more of the animal species is human, and one or more of the animal species is non-human. The human cells contained in the chimeric embryo are composed of any number of cell types, to include, but not limited to embryonic cells, embryonic stem cells, or a mixture of both cell types. The non-human cells contained in the chimeric embryo are composed of any number of cell types, to include, but not limited to embryonic cells, embryonic stem cells, or a mixture of both cell types.

The cells composing the chimeric embryo may contain one or more transgenes. The trangenes can be present in the human cells or the non-human cells. The present invention also includes any cell line developed from the chimeric embryo. In addition, the present invention includes any animal and any descendant of an animal developed from the chimeric embryo.

It is to be understood that both the foregoing general description and the following detailed description are exemplary and explanatory only, and are not restrictive of the invention as claimed. The following preferred embodiments, which are incorporated herein by reference, and which constitute a part of this specification, describe certain embodiments of the invention, and together with the detailed description serve to explain the principles of the present invention.

### DETAILED DESCRIPTION OF THE PREFERRED EMBODIMENTS

Reference will now be made in detail to the preferred embodiments of the present invention. Preferred methods of practicing the novel innovation are illustrated in the following paragraphs.

The embryos of the invention will be constructed by standard methods for the generation of chimeric embryos, described in references cited above, and in Robertson, E. J., ed. (1987) Teratocarcinoma and Embryonic Stem Cells: A Practical Approach (IRL, Oxford), or by any other methods which are either now currently known or unknown. In the preferred embodiment, an intact blastocyst (early embryo) of a non-human species is injected with one or more blastomeres from a four- or eight-cell human embryo; as an alternative preferred embodiment, the non-human blastocyst is injected with one or more cells from a human ES cell line.

The inventor believes, as an alternative preferred embodiment, that chimeric embryos derived entirely from human and non-human ES cells can be constructed. One way that this can be accomplished is by following the protocol developed by Nagy, et al., to produce ES cell-derived mouse embryos. The first step is to generate human or non-human “tetraploid” embryos (i.e., embryos having twice the normal number of chromosomes in each of their cells) by electric pulse-mediated fusion of normal two-cell embryos. Clumps of 10-15 ES cells, comprising a mixture of human and non-human cells, may be sandwiched between two tetraploid embryos in special culture wells, and cultured overnight or longer.

The preferred method, alternative method, or any additional method either now known or unknown, of creating human/non-human chimeras will be used to create the following alternative embodiments of the present invention.

It will be apparent to persons of ordinary skill in the art that modifications and variations can be made in the method of forming chimeric embryos and chimeric animals, and in the particular types of chimeric embryos and animals that are formed, without departing from the scope or spirit of the invention. For example, in the embodiments discussed in this application, various methodologies and four specific end points are identified. Numerous additional methodologies and numerous additional end points are possible. By means of example, alternative methods are currently being researched and may in the future be developed, that will enable the formation of a chimeric embryo or animal. In addition, the variety of research models is limited only by the creativity and imagination of the researcher. Thus, it is intended that the present invention cover the modifications and variations of the invention, provided they come within the scope of the appended claims and their equivalents.

1. Chimeric Mouse/human Embryos for Studies in Developmental Biology

Mouse or human blastomeres aggregated with blastomeres or ES cells of other species will be stored frozen for use in experimental studies of embryonic development. These two species are physiologically and anatomically dissimilar because of their phylogenetic distance within the category of mammals. It is therefore expected that chimeras of their embryonic cells will develop cooperatively to only a limited extent in vitro, or, if implanted into mouse foster mothers, in utero. Nonetheless, the extent to which such cells can cooperate in the formation of an embryo is of great interest to scientists working in the field of developmental biology. The opportunity to increase the capacity of such chimeras to undergo further development by the introduction into the blastomeres of one species genes derived from the other species, or alternatively, by the specific inactivation of genes that may determine such developmental incompatibility, make this invention an experimental system of great utility and convenience to these scientists.

2. Chimeric Baboon/human or Chimpanzee/human Embryos for Developmental Toxicology Assays

Chimeras of human and nonhuman primate blastomeres or blastomeres and ES cells are expected to progress to at least the fetal stages of development, by virtue of the fact that these species are physiologically and anatomically similar, and phylogenetically close within the category of mammals. Such embryos can be constructed to contain fewer than 50% human cells, and can be permitted to develop in vitro or in utero in primate foster mothers. This system would be of great utility to pharmaceutical companies and chemical manufacturers who wish to determine the teratogenicity and developmental toxicity of compounds under development for medical, industrial or consumer use. The developing embryos can be exposed to drugs or chemical compounds via the tissue culture medium or the maternal circulation, and effects on developmental outcome can be assessed morphologically and histologically. It is of particular interest to determine whether the tissues of human origin had a differential susceptibility to the test compounds. The non-human status of the chimeric primate embryos would make them a particularly favorable assay system for these purposes.

3. Chimpanzee/human Chimeras for Studies in Cardiovascular Physiology

Chimpanzee/human chimeric embryos can be gestated in chimpanzee or human foster mothers. Those organisms that progress to the fetal stage can be used as a source of hearts for physiological studies of the effects of ventricular stress on the breakdown and repair of heart proteins in vitro. Differential effects on the human and nonhuman components of the chimeric hearts can be assessed. This would provide important data, not previously obtainable with human tissues, on cardiac damage, and provide a system for evaluation of drugs that might be protective against tissue damage under conditions of stress, such as hypertension. If developmental incompatibilities between chimpanzee and human blastomeres or ES cells can be experimentally overcome, these chimeric fetuses may eventually be brought to term. Such chimeric animals will provide ideal test systems for cardiovascular effects of whole animal stress, such as treadmill exercises, hypothermia, etc. The ability thus afforded to evaluate effects on human heart tissues in the intact organism would be unprecedented.

4. Chimpanzee/human and Pig/human Chimeras as a Source of Hearts for Transplantation to Cardiac Patients

As barriers to developmental compatibility between chimpanzees and humans are surmounted (as they already have been for the more distantly related sheep and goat) it will be possible to bring chimeras between these species to term. The hearts of these chimeras can be used in medically indicated transplantations with the relatively high expectation that they will not be rejected by the human host, as xenotransplants of hearts from baboons to humans have inevitably been. The possibility of using blastomeres from donated human embryos in conjunction with chimpanzee ES cells can obviate the potential difficulties associated with scarcity of chimpanzees. Indeed, as described above, it will eventually prove possible to produce chimeras that are completely ES cell-derived. Currently, pigs are being bred and used for xenotransplantation of hearts to human cardiac patients, as pig hearts are recognized as being anatomically and physiologically similar to human hearts. Germ line genetic engineering of the pigs is now being attempted in order to curtail immunological rejection of the hearts, but with only limited success. Using the invention described here, it will eventually prove possible to bring pig/human chimeras to term and thus have a non-human source of hearts that contain some human tissue and are more likely to be tolerated by human patients than hearts of purely non-human origin.

These examples are not intended to be an exhaustive list of embodiments of this invention. It will be apparent to those skilled in the art that various modifications and variations can be made in the creation of the chimeric embryos and chimeric animals of the present invention without departing from the scope or spirit of the invention. For example, in the embodiments mentioned above, various changes may be made to host or donor cell types or the origin of those cells without departing from the scope and spirit of the invention. Further, it may be appropriate to make additional modifications or changes to the culture and propagation of these chimeric embryos and chimeric animals without departing from the scope of the invention. Thus, it is intended that the present invention cover the modifications and variations of the invention provided they come within the scope of the appended claims and their equivalents.

I claim:

1. A chimeric embryo comprising cells from a first and one or more second animal species, wherein said first animal species is human, wherein said second animal species is non-human, and wherein said second animal species is non-primate.

2. The chimeric embryo according to claim 1, wherein said cells from said first animal species are embryonic cells.

3. The chimeric embryo according to claim 1, wherein said cells from said first animal species are embryonic stem cells.

4. The chimeric embryo according to claim 1, wherein said cells from said first animal species are comprised of a mixture of both embryonic cells and embryonic stem cells.

5. The chimeric embryo according to claim 1, wherein said cells from said second animal species are embryonic cells.

6. The chimeric embryo according to claim 1, wherein said cells from said second animal species are embryonic stem cells.

7. The chimeric embryo according to claim 1, wherein said cells from said second animal species are comprised of a mixture of both embryonic cells and embryonic stem cells.

8. The chimeric embryo according to claim 1, wherein one or more said cells from said first animal species comprises one or more transgenes.

9. The chimeric embryo according to claim 1, wherein one or more said cells from said second animal species comprises one or more transgenes.

10. A cell line developed from a chimeric embryo comprising cells from a first and one or more second animal species, wherein said first animal species is human, wherein said second animal species is non-human, wherein said second animal species is non-primate, wherein said cells from said first animal species are selected from among the group comprising: embryonic cells; embryonic stem cells; and a mixture of both embryonic cells and embryonic stem cells, and wherein said cells from said second animal species are selected from among the group comprising: embryonic cells; embryonic stem cells; and a mixture of both embryonic cells and embryonic stem cells.

11. The cell line according to claim 10, wherein one or more of said cells from said first animal species comprises one or more transgenes.

12. The cell line according to claim 10, wherein one or more of said cells from said second animal species comprises one or more transgenes.

13. An animal developed from a chimeric embryo comprising cells from a first and one or more second animal species, wherein said first animal species is human, wherein said second animal species is non-human, wherein said second animal species is non-primate, wherein said cells from said first animal species are selected from among the group comprising: embryonic cells; embryonic stem cells; and a mixture of both embryonic cells and embryonic stem cells, and wherein said cells from said second animal species are selected from among the group comprising: embryonic cells; embryonic stem cells; and a mixture of both embryonic cells and embryonic stem cells.

14. The animal according to claim 13, wherein one or more of said cells from said first animal species comprises one or more transgenes.

15. The animal according to claim 13, wherein one or more of said cells from said second animal species comprises one or more transgenes.

16. A descendant of said animal of claim 13.

17. A descendant of said animal of claim 14.

18. A descendant of said animal of claim 15.

19. A chimeric embryo comprising cells from a first and one or more second animal species, wherein said first animal species is human, and wherein said second animal species is murine.

20. The chimeric embryo according to claim 19, wherein said cells from said first animal species are embryonic cells.

21. The chimeric embryo according to claim 19, wherein said cells from said first animal species are embryonic stem cells.

22. The chimeric embryo according to claim 19, wherein said cells from said first animal species are comprised of a mixture of both embryonic cells and embryonic stem cells.

23. The chimeric embryo according to claim 19, wherein said cells from said second animal species are embryonic cells.

24. The chimeric embryo according to claim 19, wherein said cells from said second animal species are embryonic stem cells.

25. The chimeric embryo according to claim 19, wherein said cells from said second animal species are comprised of a mixture of both embryonic cells and embryonic stem cells.

26. The chimeric embryo according to claim 19, wherein one or more said cells from said first animal species comprises one or more transgenes.

27. The chimeric embryo according to claim 19, wherein one or more said cells from said second animal species comprises one or more transgenes.

28. A chimeric embryo comprising cells from a first and one or more second animal species, wherein said first animal species is human, and wherein said second animal species is selected from among the group comprising domestic pig, mouse, rat, and rabbit.

29. The chimeric embryo according to claim 28, wherein said cells from said first animal species are embryonic cells.

30. The chimeric embryo according to claim 28, wherein said cells from said first animal species are embryonic stem cells.

31. The chimeric embryo according to claim 28, wherein said cells from said first animal species are comprised of a mixture of both embryonic cells and embryonic stem cells.

32. The chimeric embryo according to claim 28, wherein said cells from said second animal species are embryonic cells.

33. The chimeric embryo according to claim 28, wherein said cells from said second animal species are embryonic stem cells.

34. The chimeric embryo according to claim 28, wherein said cells from said second animal species are comprised of a mixture of both embryonic cells and embryonic stem cells.

35. The chimeric embryo according to claim 28, wherein one or more said cells from said first animal species comprises one or more transgenes.

36. The chimeric embryo according to claim 28, wherein one or more said cells from said second animal species comprises one or more transgenes.
